(19) United States
(12) Patent Application Publication
(10) Pub. No.: US 2025/0270495 A1
(43) Pub. Date: Aug. 28, 2025

(54) SYSTEMS AND METHODS FOR MICROBIAL BIOMASS PRODUCTION

(71) Applicant: C1Pro Inc., Lewes, DE (US)

(72) Inventors: Irina Nizovtseva, Lewes, DE (US); Mikhail Shumakov, Lewes, DE (US); Nikolai Kuznetsov, Lewes, DE (US); Dmitrii Chernushkin, Lewes, DE (US); Antoine Mendelovici, Lewes, DE (US)

(21) Appl. No.: 19/200,153

(22) Filed: May 6, 2025

Related U.S. Application Data
(63) Continuation of application No. PCT/US2023/079478, filed on Nov. 13, 2023.
(60) Provisional application No. 63/383,596, filed on Nov. 14, 2022.

(51) Int. Cl. C12M 1/36; C12M 1/34

(52) U.S. Cl. CPC C12M 41/48; C12M 41/12; C12M 41/26; C12M 41/34; C12M 41/40

(57) ABSTRACT

The present disclosure relates to embodiments for solving the problem of microbial biomass production. The present disclosure describes systems and methods that increase the productivity of microbial biomass production by modeling the process of producing the specified biomass and selecting the parameters of the process of producing microbial biomass based on the results of this modeling. Also disclosed are systems and methods that reduce the resources used to produce microbial biomass by modeling the production process of this biomass using a process model improved by retraining.

### CROSS-REFERENCE TO RELATED APPLICATIONS

The present application is a continuation of PCT Patent Application No. PCT/US2023/079478, filed Nov. 13, 2023, which claims the benefit of priority of U.S. Provisional Application No. 63/383,596, filed Nov. 14, 2022, each of which is incorporated by reference in its entirety herein.

### BACKGROUND

Embodiments provided herein relate to control systems, and more specifically to systems and methods for producing microbial biomass.

The current market forces microbial biomass producers to increase the productivity and efficiency requirements of producing microbial biomass. This, in turn, leads to the fact that producers have to pay attention to both the safety of production and the efficiency of expending used resources.

These tasks require an enhanced control over the process of microbial biomass production, which leads to the partial or complete exclusion of a human controller from the process of microbial biomass production. Human controllers are replaced by automatic, highly intelligent tools.

The active development of biotechnology science and usage extension of devices or installations operating on the basis of various types of physico-chemical reactions challenges the research community. Current research focuses on determining the necessity of creating unified trainable digital systems that include a set of methods and mathematical models describing the full cycle of operation of devices (installations) throughout the entire life cycle, including design, testing, production, and operation. The rapid development of analytical cloud services and the Internet turns the request for a virtual environment that emulates the full cycle of the microbial biomass production process from a conceptual trend into an urgent need and an obligatory next step of technological development.

Over the past five years, the development and implementation demand for digital copies of physical objects and processes has grown significantly. For example, companies are interested in increasing productivity, costs optimization, and increasing business efficiency. Virtual prototypes of its real components provide an opportunity for more competent design of installations and testing of systems, a quick response in the case of a process running outside the regulations (including the identification of physical and technological problems) and finally producing better products and services with a reasonable minimum amount of costs, including due to the predictive power of the virtual environment, based on a structured matrix of requirements for equipment and process characteristics, economic and environmental indicators, technical documentation, result standards, and its components.

In parallel with the development of technological hardware solutions and bioreactor devices, the volume of software running on these devices has also grown rapidly. However, they require excessive computing resources to work or have low efficiency and reliability.

Effective management of the bioreactor operation requires timely forecasting of the biomass production process, taking into account the geometric and hydrodynamic characteristics of the devices, conducting timely proactive analysis with high efficiency of detecting prerequisites, and violations of the microbial biomass production process, using sensor data and the computing resources of the system provided for this purpose.

For example, patent application CN108681297A discloses a technology of real-time control of a biological fermentation process using a system of sensors that collect information during the fermentation process such as pressure, temperature, pH level, oxygen concentration, etc., and controls these parameters based on the data obtained, to achieve an optimal environment for microorganism production.

The technology described above copes well with the task of fermentation process controlling in real time, but this technology is able to respond only to changes in the monitored parameters, due to real-time operation with weak predictive capability.

Embodiments disclosed herein solve the problem of microbial biomass production with optimal productivity and efficiency by using the results of modeling this process.

### SUMMARY

The technical solution is intended for the production of microbial biomass.

The technical result of claimed technical solution is to increase the productivity of microbial biomass production by the way of modeling the production process of the specified biomass and selecting the optimal parameters of the microbial biomass production process based on the results of this modeling.

Another technical result of this technical solution is to reduce the resources used to produce microbial biomass by modeling the process of producing this biomass using a model process improved by retraining.

In some embodiments, these technical results are achieved by using a microbial biomass producing system, which includes: a process emulation tool configured to emulate in a virtual environment the biomass production process with specified parameters, where the virtual environment is a model of industrial production of microorganisms; a selection tool is configured to select at least one control tool for the biomass production process from a list of controls using a trained process model based on the result of emulation of the biomass production process, while the model process is a set of rules for controlling the biomass production process; determining the parameters of the selected control tool using a trained model process based on the results of the emulation of the biomass production process; and/or a production facility designed to produce biomass.

Some embodiments provided herein relate to a microbial biomass production system. In some embodiments, the microbial biomass production system includes methanotrophic microorganisms.

In some embodiments, parameters of the biomass production process include productivity of biomass production; the proportion of crude protein contained in the biomass; specific power consumption; and/or the specific consumption of the resources used to produce biomass.

In some embodiments, the result of the emulation of the biomass production process includes at least information about the resources of the management tools used to produce the specified biomass and/or parameters of the biomass production process.

In some embodiments, the control tool includes at least a regulating gas mixture flow tool configured to control the gas mixture used in the process of producing biomass, a controlling biomass tool intended to control the parameters of the resulting biomass, and/or an aeration tool intended to ensure the mass transfer of the nutrient medium gas components and culture liquid oxygen.

In some embodiments, the gas mixture is controlled by at least changing the composition of the gas mixture by changing the concentration of the components of the gas mixture, changing the temperature of the gas mixture, and/or changing the pressure of the gas mixture.

In some embodiments, the gas mixture is controlled in a predetermined explosion-proof area in such a way as to ensure the maximum permissible concentration of oxygen.

In some embodiments, at least two components of the gas mixture act as nitrogen, oxygen, natural gas, carbon dioxide, and/or air.

In some embodiments, the characteristics of the increased oxygen flow zone according to the Gibbs-Rosebum diagram act as parameters of the gas mixture flow control device operation.

In some embodiments, the characteristics of the increased oxygen flow zone according to the Gibbs-Rosebum diagram includes concentrations of nitrogen at 81.9%, methane at 6.0%, and oxygen at 12.1%.

In some embodiments, the biomass control includes at least changing the biomass temperature and/or changing the pH of the biomass.

In some embodiments, the aeration control includes a change in the variable aeration, which is reached at the maximum value of the enzyme that is the activator of molecular oxygen in the ventilation zone, followed by oxidation of the organic substrate.

In some embodiments, a training tool intended to retrain the model process in such a way that at least: for the specified parameters of biomass production, the specified biomass will be produced with less usage of management resources; and/or for the specified resources of the biomass production management tools, more optimal parameters for producing the specified biomass will be selected.

These technical results are achieved by using microbial biomass production method, which is implemented using the microbial biomass production system and includes at least the following stages: a) the biomass production process with specified parameters is emulated in a virtual environment, where the virtual environment is a model of industrial production of microorganisms; b) selection of at least one controlling tool of the biomass production process from the list of control tools using a trained model process based on the result of emulation of the biomass production process, while the model process is a set of rules for controlling the biomass production process; c) operation parameter determination of the selected control tool using a trained process model based on the results of emulation of the biomass production process; d) biomass production using the selected control tool that works with certain parameters.

In some embodiments, the microorganism is a methanotrophic microorganism.

In some embodiments, the parameters of the biomass production process include at least: productivity of biomass production; the proportion of crude protein within the biomass; specific power consumption; and/or the specific consumption of the resources used to produce biomass.

In some embodiments, the result of the emulation of the biomass production process includes at least: information about the resources of the management tools used to produce the specified biomass; and/or parameters of the biomass production process.

In some embodiments, the control tool includes at least: a regulating gas mixture flow tool, configured to control the gas mixture used in the process of producing biomass; a controlling biomass tool, intended to control the parameters of the resulting biomass; and/or an aeration tool, intended to ensure the mass transfer of the nutrient medium gas components and culture liquid oxygen.

In some embodiments, the gas mixture is controlled by at least: changing the composition of the gas mixture by changing the concentration of the components of the gas mixture; changing the temperature of the gas mixture; and/or changing the pressure of the gas mixture.

In some embodiments, at least two components of the gas mixture include nitrogen, oxygen, natural gas, carbon dioxide, and/or air.

In some embodiments, the gas mixture is controlled in a predetermined explosion-proof area in such a way as to ensure the maximum permissible concentration of oxygen.

In some embodiments, the characteristics of the increased oxygen flow zone according to the Gibbs-Rosebum diagram act as parameters of the gas mixture flow control device operation.

In some embodiments, the characteristics of the increased oxygen flow zone according to the Gibbs-Rosebum diagram includes concentrations of nitrogen at 81.9%, methane at 6.0%, and oxygen at 12.1%.

In some embodiments, the biomass control includes at least changing the biomass temperature and/or changing the pH of the biomass.

In some embodiments, the aeration control includes a change in the variable aeration, at which is reached the maximum value of the enzyme that is the activator of molecular oxygen in the ventilation zone, followed by oxidation of the organic substrate.

In some embodiments, a training tool intended to retrain the model process in such a way that at least: for the specified parameters of biomass production, the specified biomass will be produced with less usage of management resources; and/or for the specified resources of the biomass production management tools, more optimal parameters for producing the specified biomass will be selected.

### BRIEF DESCRIPTION OF THE DRAWINGS

FIG. 1 represents an example of a block diagram of a microbial biomass production system.

FIG. 2 represents an example of a block diagram of a method of producing microbial biomass.

FIG. 3 represents an exemplary Gibbs-Rosebum diagram.

FIG. 4 represents an example of a block diagram of emulation process tool of microbial biomass production.

FIG. 5 represents an example of a general-purpose computer system.

Although the technical solution may have various modifications and alternative forms, the characteristic features shown as an example in the drawings will be described in detail. The purpose of the description does not limit the technical solution to its specific implementation. The purpose of the description is to cover all changes and modifications that fall within the scope of this technical solution, as defined in the claims.

### DETAILED DESCRIPTION

The objects and features of embodiments described herein, including ways to achieve these objects and features will become obvious by referring to the exemplary embodiments provided herein. However, is the disclosure is not limited to the exemplary embodiments disclosed herein, and various additional embodiments are included in view of the disclosure provided herein. The essence given in the description is nothing more than specific details necessary to help a specialist qualified in this field of technology to understand this technical solution, which is determined in the claims.

Definitions and concepts are provided herein, which will be used throughout the description.

A virtual environment has its ordinary meaning as understood in light of the specification, and refers to a specially allocated (isolated) environment, which may include a set of computing resources or their logical combination, abstracted from a hardware implementation and providing logical isolation from each other of computing processes running on the same physical resource.

An exemplary purpose of a virtual environment is, for example, to perform emulation (or modeling) of physical processes. The goal is to reproduce the behavior as accurately as possible, in contrast to various parameters of physical processes, for example, the production of microbial biomass, which includes reproducing the operation of various means of the technological cycle. The term “emulate” as used herein refers to the ability of a program or device to imitate the operations of another program or device, for example, to imitate a physical process. A process emulation tool is a tool that is configured to emulate or imitate a physical process in a virtual environment.

An artificial neural network has its ordinary meaning as understood in light of the specification, and refers to a system of simple processors (artificial neurons) connected and interacted with each other. Such processors are usually quite simple (especially in comparison with processors used in personal computers). Each processor of such a network deals only with the signals that it periodically receives, and the signals that it periodically sends to other processors. Nevertheless, being connected to a fairly large network with controlled interaction, such individually simple processors together are able to perform quite complex tasks.

From the machine learning point of view, the use of a neural network is a special case of pattern recognition methods, discriminant analysis, and/or clustering methods, for example.

Machine learning (ML, machine learning) has its ordinary meaning as understood in light of the specification and refers to a class of artificial intelligence methods, which characteristic features is not the direct solution of a problem, but learning in the process of applying solutions to many similar problems. For the construction of such methods, the means of mathematical statistics, numerical methods, optimization methods, probability theory, graph theory, various techniques for working with data in digital form are used.

There are three types of training: case-based learning, or inductive learning, is based on the identification of empirical patterns in the data; deductive learning involves the formalization of experts' knowledge and their transfer to a computer in the form of a knowledge base; and reinforcement learning based on the trial and error method with the encouragement of correct actions in the current situation.

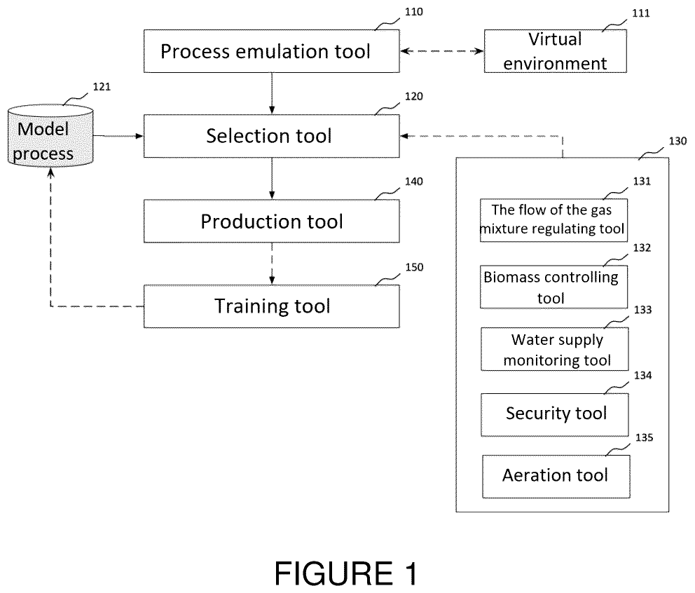

FIG. 1 represents a structural diagram of the microbial biomass production system.

Block diagram of the microbial biomass production system includes a process emulation tool 110, a virtual environment 111, a selection tool 120, control tools 130, a gas mixture flow control tool 131, a biomass control tool 132, a water supply control tool 133, a safety tool 134, an aeration tool 135, a production tool 140, a training tool 150.

The process emulation tool 110 is configured to emulate in the virtual environment 111 the biomass production process with specified parameters, where the virtual environment 111 is a model of industrial production of microorganisms.

In some embodiments, the microorganism is a methanotrophic microorganism.

In some embodiments, at least two parameters of the biomass production process include: productivity of biomass production; the proportion of crude protein contained in the biomass; specific power consumption; and/or the specific consumption of resources used to produce biomass.

In some embodiments, at least the following are the results of the emulation of the biomass production process: information about the resources of the management tools 130 used to produce the specified biomass; parameters of the biomass production process.

In some embodiments, the usage of preliminary emulation of the biomass production process (online mode) in the virtual environment 111 contributes at least: improving the efficiency, stability and productivity of the biomass production process during the subsequent operation of a set of tools that provide a full cycle of the process (including the stages of substrate preparation, inoculation, cultivation, separation, and plasmolysis); guarantees of achieving the parameters of biomass production for emulation in the physical (offline mode) process of biomass production; improving the fault tolerance of the production tool 140; ensuring the continuity of the biomass production process through predictive detection of anomalies and elimination of causes, reducing the risks of disruption of biomass production.

This result is achieved through the usage of a single digital platform that connects the virtual environment 111 and the emulation tool 110 with the rest of the control tools 130 of the biomass production process and the biomass production tool 140, as well as providing a way to control the process of producing biomass, which is a comprehensive software solution containing various groups of models based on the principle of the main stages of designing the system operation and the course of the biomass production process, including sets of at least: models in 1D and 3D formats, including geometric and hydrodynamic parameters of devices that provide a full cycle of the biomass production process (bioreactor, etc.), allowing to model the main and auxiliary structures and evaluate the capabilities and features of design solutions of the production tools 140; 3D models complex that allow to conduct tests that actually correspond to full-scale experiments and the flow of the technological process in the operation of the production tool 140 (for example, a bioreactor); mathematical models of physico-chemical processes that determine the sequence and segment the biomass production process into subprocesses characterized by physical processes and chemical reactions and transformations in accordance with the matrix of efficiency and safety requirements, including a mathematical model of mass transfer in a heterogeneous medium of a jet fermenter, developed for dispersed and continuous phases, including modules describing motion, mass transfer, energy transfer, turbulence; methods of processing data received from sensors of control tools 130 and production tool 140; storing data tools on the progress and operating modes of control tools 130 and production tool 140, input and output parameters of subprocesses (including preparation of substrates, inoculation, cultivation, separation, plasmolysis); analytical center for decision-making (algorithms based on a comparative analysis of data from real sensors coming from control tools 130, production tool 140 and virtual sensors readings of the virtual environment 111, which allows you to identify anomalies and determine the causes of their occurrence.

The selection tool 120 is configured for: selection at least one control tool for the biomass production process (hereinafter, the control tool) from the list of control tools 130 using a trained model process 121 based on the result of emulation of the biomass production process, while the model process 121 is a set of rules for controlling the process of producing biomass; determination of the operation parameters of the selected control tool using a trained model process 121 based on the emulation results of the biomass production process.

In some embodiments, the control model 121 is a pre-trained neural network.

In some embodiments, the control model 121 is trained in advance on real biomass production processes.

In some embodiments, the control model 121 is trained in advance with a teacher (such as English supervised learning) under the control of an operator who corrects the errors of the control model 121 that occur during the training process.

In some embodiments, the control tools 130 include at least: flow of a gas mixture regulating tool 131, configured to control the gas mixture used in the biomass production process; biomass controlling tool 132, configured to control the parameters of the received biomass; water supply monitoring tool 133, configured to control the water supply of the production tool 140; security tool 134, configured for security, fire alarm, video surveillance, etc.; and/or aeration tool 135, configured to provide mass transfer of gas components of the nutrient medium and oxygen of the culture liquid.

In some embodiments, the control tools 130 carry out at least: fully automatic control of the relevant parameters and stages of the biomass production process; and/or automatic monitoring of the relevant parameters and stages of the biomass production process with confirmation of the selected actions by the operator.

In some embodiments of the system, the control tools 130 include a variety of sensors that allow collection of data about the biomass production process. Based on the collected data, the control tools 130 allows at least: to control the process of biomass production based on pre-set operating parameters for the specified tool; adjust the pre-set parameters of the specified tool to match the parameters of the biomass production process; and/or transfer the collected data to the training tool 150 for retraining the process model 121.

In some embodiments, the gas mixture is controlled in a predetermined explosion-proof area in such a way as to ensure the maximum permissible oxygen concentration.

In some embodiments, at least one of the gas mixture control functions includes: changing the composition of the gas mixture by changing the concentration of the components of the gas mixture; changing in the temperature of the gas mixture; and/or changing in the pressure of the gas mixture and dissolved gases.

For example, temperature is an important parameter of fermentation, because during the cultivation of many microorganisms, a temperature deviation of a couple of degrees can lead to a significant decrease in the productivity of biomass growth. The cultivation temperature is maintained with an accuracy not less than +0.5° C.

In another example, the partial pressure of dissolved oxygen is set as percentage of saturation. The set value has a lower and upper limit, the distance between them is 10-20%.

In another example, several principles of regulating the partial pressure of dissolved oxygen are used, including: the change of the speed rotation of the agitator, proportional to the change in the partial pressure of dissolved oxygen, carried out by the regulating the flow of the gas mixture regulating tool 131; combining changes in the speed of rotation of the agitator of the compressed air amount to the production tool 140, carried out by the means of regulating the flow of the gas mixture regulating tool 131; the addition of a substrate or some of its components (it is believed that the partial pressure of dissolved oxygen is inversely proportional to the intensity of fertilization, fertilization is usually performed using controlled peristaltic pumps), carried out by controlling the biomass tool 132.

Any of the above methods may be combined with each other in any variety of combinations.

In some embodiments, at least two components of the gas mixture act as components of the gas mixture, including: nitrogen; oxygen; natural gas; carbon dioxide; and/or air.

In some embodiments, the characteristics of the increased oxygen flow zone according to the Gibbs-Rosebum diagram act as parameters of the operation of the gas mixture flow control tool 131.

In some embodiments, the characteristics of the increased oxygen flow zone according to the Gibbs-Rosebum diagram are concentrations of nitrogen at 81.9%, methane at 6.0%, and oxygen at 12.1%.

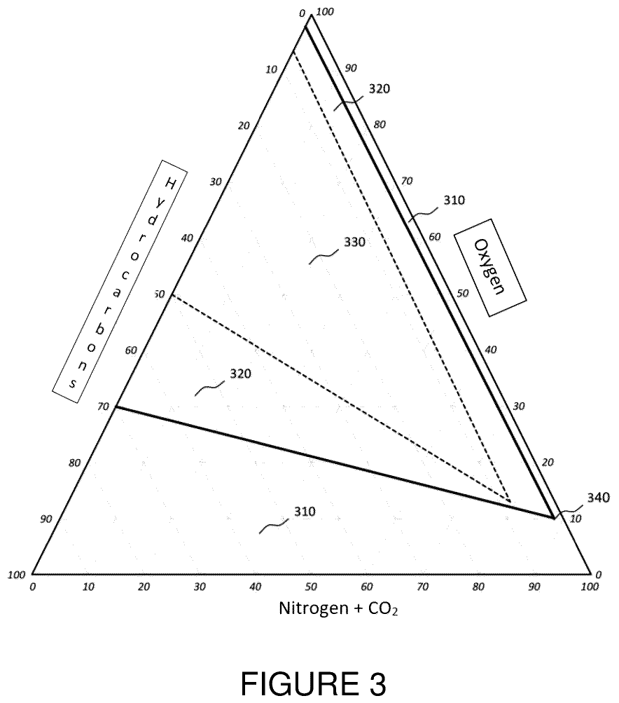

As shown in FIG. 3, in some embodiments, the Gibbs-Rosebum diagram identifies a non-explosive region 320 with an oxygen concentration in the gas mixture of up to 20-25%, a working process which allows increasing the productivity of the process up to 2-2.5 times.

In some embodiments, the flow of the gas mixture regulating tool 131 includes sensors for analyzing the gas mixture content of methane, oxygen, carbon dioxide, and nitrogen.

In some embodiments, the flow of a gas mixture regulating tool 131 provides a concentration of oxygen in the gas mixture of no more than 8% during the production of biomass, which in turn significantly reduces the area of the safe work area (as shown in FIG. 3:330) (by about 2-2.5 times) and prevents an increase in the productivity of the growth process of microorganisms.

In some embodiments, the parameters of the operation of the biomass controlling tool 132 are at least: parameters of biomass mixing (rotation modes); and/or parameters of mass transfer of the culture solution.

In some embodiments, at least one of the following acts as a biomass management: change in biomass temperature; and/or change in the pH of the biomass.

For example, the pH change is based on comparison of the specified upper and lower pH values with the actual pH value. Automatic regulation of the pH value is provided by peristaltic pumps, which respectively dose acid or base. The pH measurement must be accurate (+0.02 pH units), because pH changes carry important information about the kinetics of the process.

In some embodiments, the aeration control is a change in the aeration variable, at which the maximum value of the enzyme NAD·H₂ (nicotinamide adenine dinucleotide) is reached, which is an activator of molecular oxygen in the aeration zone with subsequent oxidation of the organic substrate.

In some embodiments, the control tools 130 contains a single control tool (for example, the flow of a gas mixture regulating tool 131, the biomass controlling tool 132). In this case, the selection tool only determines the parameters of the specified (only) control tool.

In some embodiments, the production tool 140 is configured to produce biomass using one or more of the selected control tools 130, operating with certain parameters.

In some embodiments, the production tool 140 is a bioreactor.

In some embodiments, at least the following stages of the technological process of biomass production are performed in the production tool 140: dry and liquid chemicals reception and storage; solutions preparation of nutrient salts of micro and macronutrients; seeded biomass cultivation; bioprotein production; biomass thickening; biomass inactivation; bioprotein drying; granulation and packaging; bioprotein storage; and/or sterilization and biological treatment of wastewater.

In some embodiments, the specified stages of the technological biomass production process are controlled by the control tools 130, which includes collecting data from the sensors of the control tools 130, analyzing them and changing the parameters of those processes controlled by the corresponding control tools 130.

In some embodiments, the control tools 130 and the production tool 140 are interconnected at least: directly, using any hardware and software tools known from the state of the art; local computer network; a global computer network (for example, the Internet).

In some embodiments, if the control tools 130 and the production tool 140 are interconnected through a global computer network, they form a cloud managed by cloud technologies known from the state of the art.

For example, the separation of seeded biomass (as a component of the production tool 140) may contain laboratory fermenters, FKER-1M, MPU UFS 32, MPU emk. XZB, the server of the factory dispatching console, connected via the global computer network Internet with the control tools 130 controlled by the operator of the seeding biomass separation.

In another example, the separation of storage and preparation of solutions of nutrient salts and trace elements (as a component of the production tool 140) may contain counters, terminals, USO, MPU, a server of the central factory control panel connected via a local Ethernet computer network with control tools 130 controlled by the operator of the department of storage and preparation of solutions of nutrient salts and trace elements.

In some embodiments, the training tool 150 is used to retrain the model process 121 in such a way that at least: for the specified parameters of biomass production, the specified biomass will be produced with less use of management resources; for the specified resources of the biomass production management tools, more optimal parameters for producing the specified biomass will be selected.

In some embodiments, the retraining of the model process 121 occurs on the basis of data received from the selected control tools 130 in the biomass production process.

In another variant of the system implementation, the model process 121 is retrained based on the response from the operator correcting the operation of the control tools 130.

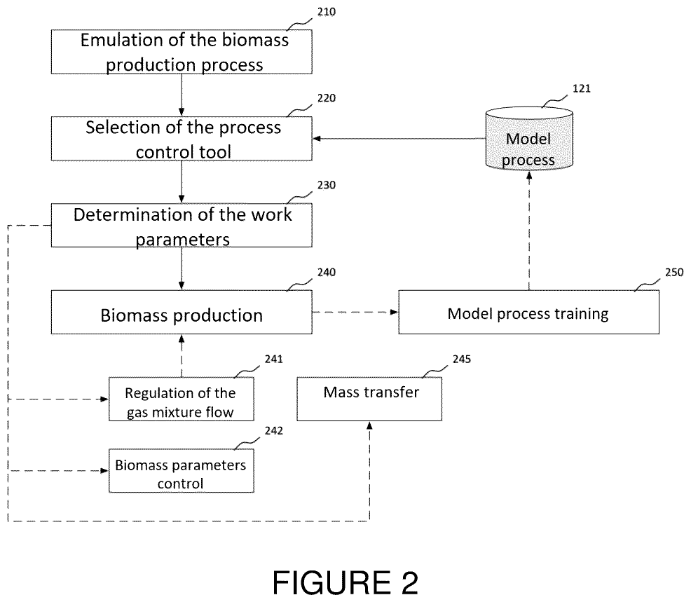

FIG. 2 represents a structural diagram of an exemplary microbial biomass production method.

Block diagram of a microbial biomass production method (hereinafter, biomass) includes stage 210, at which the biomass production process is emulated, a stage 220, at which a process control tool is selected, a stage 230, at which the operation parameters are determined, a stage 240, at which biomass is produced, a stage 241, at which the flow of the gas mixture is regulated, a stage 250, at which a model process is trained.

At step 210, using the emulation process tool 110, the biomass production process with specified parameters is emulated in the virtual environment 111, where the virtual environment 111 is a model of industrial production of microorganisms.

At step 220, using the selection tool 120, at least one controlling the biomass production process tool (hereinafter, the control tool) is selected from the list of controls 130 using a trained model process 121 based on the result of emulation of the biomass production process, while the model process 121 is a set of rules for controlling the biomass production process.

At step 230, using the selection tool 120, the operation parameters of the selected control tools 130 are determined using a model process 121 based on the results of the emulation of the biomass production process.

At step 240, biomass is produced using the production tool 140 using the selected control tools 130 operating with certain parameters.

In step 241, the gas mixture used in the biomass production process is controlled using the gas mixture flow control device 131.

At step 250, in addition, after producing the biomass, the model process 121 is retrained using the training tool 150 in such a way that at least: for the specified parameters of biomass production, the specified biomass will be produced with less use of management resources; for the specified resources of the biomass production management tools, more optimal parameters for producing the specified biomass will be selected.

FIG. 3 represents an exemplary Gibbs-Rosebum diagram.

In some embodiments, during the cultivation process of methane-oxidizing microorganisms, natural gas and air are supplied to the device as a carbon source. During the growth of microorganisms, carbon dioxide is released. In this case, the gas phase contains methane as a fuel, oxygen as an oxidizer, and a mixture of air, nitrogen, and carbon dioxide as an inert filler. At certain ratios, this gas phase can be explosive (region 310).

The triangular Gibbs-Rosebum diagram (FIG. 3) shows the change in the explosion limit in the fuel-oxidizer-inert component system. With an increase in the content of the inert component, the range of combustible compositions between the upper and lower concentration limits decreases. When determining the content of an inert component, both branches of the critical composition curve close up at point 340, called the cape of the explosive region.

In some embodiments, point 340 describes the composition of the explosive area near the cape: 81.9% nitrogen, 6.0% methane, and 12.1% oxygen.

In another embodiment, if the concentration of inert components increases with a fixed ratio of the contents of fuel and oxidizer in their mixture, the temperature and the value of the flame velocity decrease, since the energy of chemical transformation is spent on heating the additional components of the mixture of combustion products. This determines the dependence of the explosive limit on the content of inert components.

The productivity of the growth process of aerobic microorganisms is proportional to the amount of dissolved oxygen in the medium, which is determined by the mass exchange parameters of the fermenter and the concentration of dissolved oxygen. The concentration of dissolved oxygen in the medium, in turn, is determined by Henry's law, and is proportional to the concentration of oxygen in the gas phase.

Described on FIG. 1 system provides regulation of the concentrations of the components of the gas mixture during fermentation, which operates in the pre-required explosion-proof region 310 and at the same time provides the maximum permissible oxygen concentration.

This goal is achieved by the fact that the control tools 130 (in particular, the flow of the gas mixture regulating tool 131) receives data from the gas mixture concentration sensors (methane, oxygen, carbon dioxide, and nitrogen), processes and determines the safe mode at the maximum permissible oxygen concentration. The adjustment of the gas phase is carried out either by changing (increasing or decreasing) the supply of natural gas, or by changing (increasing or decreasing) the supply of air, or by changing (increasing or decreasing) the supply of nitrogen.

This control system for the safe operation of the fermenter allows to use concentration data of all components of the gas medium, both in the direction of decrease and in the direction of increase, which expands the scope of work in the safe zone of the working components of the gas phase, unlike the existing scheme, which provides only a limit on the concentration of oxygen.

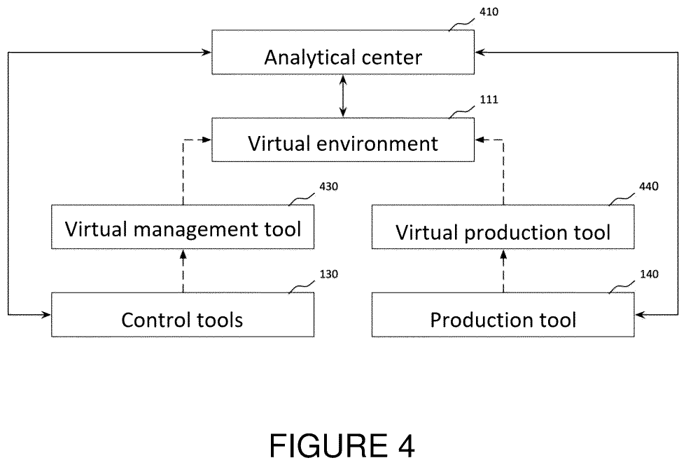

FIG. 4 represents an example of a block diagram of emulating the microbial biomass producing process.

Block diagram of emulating the microbial biomass producing process (hereinafter, biomass) includes a virtual environment 111, control tools 130, production tool 140, analytical center 410, virtual management tool 430, and virtual production tool 440.

The biomass production process control tools 130 and the biomass production tool 140 provide a full cycle of biomass production (including at the stages of substrate preparation, inoculation, cultivation, separation, plasmolysis).

The analytical center 410 is configured to analyze the data collected by the process emulation tool 110 during the simulation of the biomass production process in a virtual environment 111 and adjust the operation of the control tools 130 and the production tool 140, including through training and retraining of the model process 121.

In some embodiments, the analytical center 410 is not a single tool, but a set of individual tools combined using cloud technologies.

In some embodiments, the analytical center 410, based on the analysis of data collected by the process emulation tool 110 during the simulation of the biomass production process in a virtual environment 111, corrects the operation of virtual management tool 430 and virtual production tool 440, including through training and retraining of the model process 121.

In some embodiments, the confirmation of certain adjustments to the operation of the control tools 130 and the production tool 140 is carried out by the operator of the analytical center 410.

The virtual environment 111 contains virtual management tool 430 that emits the operation of control tools 130 and a virtual production tool 440 that emits the operation of the production tool 140.

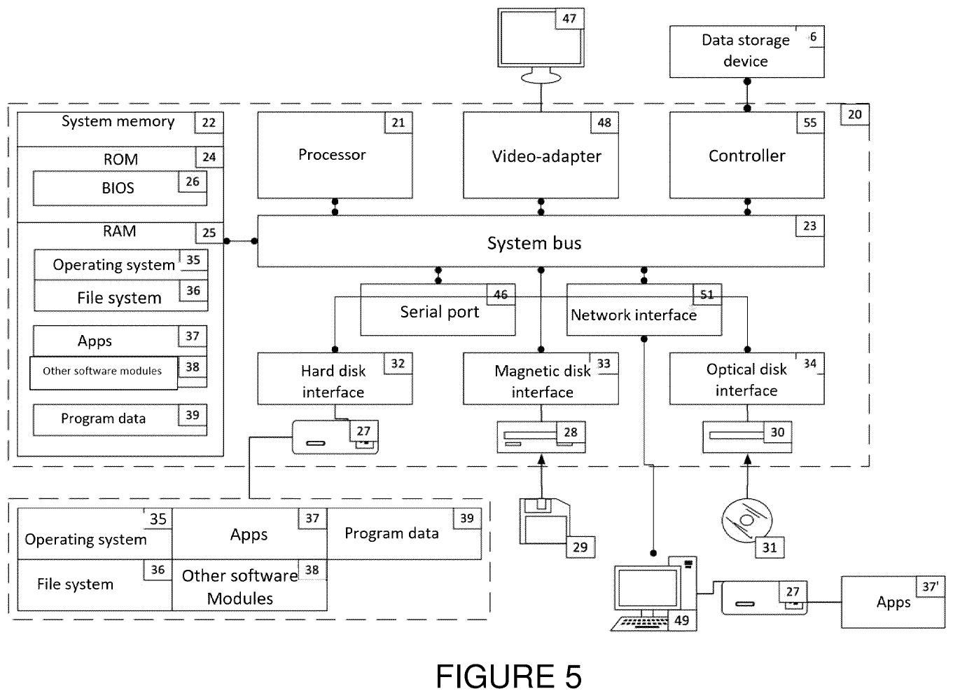

FIG. 5 represents an example of a general-purpose computer system, a personal computer or server 20 containing a central processor 21, system memory 22 and a system bus 23, which contains various system components, including memory associated with the central processor 21. The system bus 23 is implemented as any bus structure known from the state of the art, which in turn contains bus memory or a bus memory controller, a peripheral bus and a local bus that is capable to interact with any other bus architecture. The system memory contains a permanent storage device (ROM) 24, random access memory (RAM) 25. The main input/output system (BIOS) 26 contains basic procedures that ensure the transfer of information between the elements of a personal computer 20, for example, at the time of loading the operating system using ROM 24.

The personal computer 20, in turn, contains a hard disk 27 for reading and writing data, a magnetic disk drive 28 for reading and writing to removable magnetic disks 29 and an optical drive 30 for reading and writing to removable optical disks 31, such as CD-ROM, DVD-ROM and other optical media. The hard disk 27, the magnetic disk drive 28, and the optical drive 30 are connected to the system bus 23 via the hard disk interface 32, the magnetic disk interface 33, and the optical drive interface 34, respectively. Drives and corresponding computer data carriers are non-volatile means of storing computer instructions, data structures, program modules and other data of a personal computer 20.

This description reveals the implementation of a system that uses a hard disk 27, a removable magnetic disk 29 and a removable optical disk 31, but it should be understood that it is possible to use other types of computer media 56 that are able to store data in a computer-readable form (solid-state drives, flash memory cards, digital disks, random access memory (RAM), etc.) that are connected to the system bus 23 through the controller 55.

The computer 20 has a file system 36, where the recorded operating system 35 is stored, as well as additional software applications 37, other software modules 38 and program data 39. The user has the ability to enter commands and information into a personal computer 20 by input devices (keyboard 40, mouse manipulator 42). Other input devices can be used (not displayed), including, for example, microphone, joystick, game console, or scanner. Such input devices are usually connected to the computer system 20 via a serial port 46, which in turn is connected to the system bus, but can be connected in another way, for example, using a parallel port, a game port or a universal serial bus (USB). A monitor 47 or other type of display device is also connected to the system bus 23 via an interface such as a video adapter 48. In addition to the monitor 47, the personal computer can be equipped with other peripheral output devices (not displayed), for example, speakers or a printer.

The personal computer 20 is able to work in a network environment, while using a network connection with another or several remote computers 49. The remote computer (or computers) 49 are the same personal computers or servers that have most or all of the mentioned elements noted earlier when describing the essence of the personal computer 20 shown in FIG. 5. Other devices may also be present in the computer network, for example, routers, network stations, peer-to-peer devices or other network nodes.

Network connections can form a local area network (LAN) 50 and a global area network (WAN). Such networks are used in corporate computer networks, internal networks of companies and, as a rule, have access to the Internet. In LAN or WAN networks, a personal computer 20 is connected to a local network 50 via a network adapter or a network interface 51. When using networks, the personal computer 20 may use a modem 54 or other means of providing communication with a global computer network, such as the Internet. Modem 54, which is an internal or external device, is connected to the system bus 23 via serial port 46. It should be clarified that network connections are only approximate and are not required to display the exact network configuration, i.e. in reality there are other ways to establish a connection by technical tools of communication from one computer to another.

Some embodiments are provided as outlined in the following enumerated alternatives.

1. A microbial biomass production system, containing: a) a process emulation tool configured to emulate in a virtual environment of a biomass production process with specified parameters, where the virtual environment is a model of industrial production of microorganisms; b) a selection tool configured for: selecting at least one control tool of the biomass production process from a list of control tools using a trained model process based on the result of emulation of the biomass production process, wherein the model process includes a set of rules for controlling of the biomass production process; determining operation parameters of the selected control tool using a trained model process based on the results of emulation of the biomass production process; and c) a production tool configured to produce biomass using the selected control tool that operates with certain parameters.

2. The microbial biomass production system according to alternative 1, wherein the microorganism is a methanotrophic microorganism.

3. The microbial biomass production system according to any one of alternatives 1-2, wherein the parameters of the biomass production process comprise: productivity of biomass production; the proportion of crude protein contained in the biomass; specific power consumption; and the specific consumption of the resources used to produce biomass.

4. The microbial biomass production system according to any one of alternatives 1-3, wherein the result of the emulation of the biomass production process comprises: information about the resources of the management tools used to produce the specified biomass; or parameters of the biomass production process.

5. The microbial biomass production system according to any one of alternatives 1-4, wherein the control tools comprise: a flow of gas mixture regulating tool, configured to control the gas mixture used in the biomass production process; a biomass controlling tool, configured to control the parameters of the resulting biomass; or an aeration tool, configured to ensure the mass transfer of nutrient medium gas components and culture liquid oxygen.

6. The microbial biomass production system according to alternative 5, wherein the gas mixture is controlled by at least: changing the composition of the gas mixture by changing the concentration of the components of the gas mixture; changing the temperature of the gas mixture; or changing the pressure of the gas mixture.

7. The microbial biomass production system according to alternative 6, wherein at least two components of the gas mixture act as: nitrogen; oxygen; natural gas; carbon dioxide; or air.

8. The microbial biomass production system according to alternative 7, wherein the gas mixture is controlled in a predetermined explosion-proof area in such a way as to ensure the maximum permissible concentration of oxygen.

9. The microbial biomass production system according to alternative 8, wherein the characteristics of the increased oxygen flow zone according to the Gibbs-Rosebum diagram act as parameters of the gas mixture flow control device operation.

10. The microbial biomass production system according to alternative 9, wherein the characteristics of the increased oxygen flow zone according to the Gibbs-Rosebum diagram comprise nitrogen at 81.9%, methane at 6.0%, and oxygen at 12.1%.

11. The microbial biomass production system according to any one of alternatives 5-10, wherein the biomass control comprise: changing the biomass temperature; changing the pH of the biomass.

12. The microbial biomass production system according to any one of alternatives 5-11, wherein the aeration control is a change in the variable aeration, which is reached at the maximum value of the enzyme that is the activator of molecular oxygen in the ventilation zone, followed by oxidation of the organic substrate.

13. The microbial biomass production system according to any one of alternatives 1-12, further comprising a training tool configured to retrain the model process in such a way that at least: for the specified parameters of biomass production, the specified biomass will be produced with less usage of management resources; or for the specified resources of the biomass production management tools, more optimal parameters for producing the specified biomass will be selected.

14. A method of microbial biomass production, comprising: a) emulating in a virtual environment a biomass production process with specified parameters, wherein the virtual environment is a model of industrial production of microorganisms; b) selecting at least one control tool of the biomass production process from the list of control tools using a trained model process based on the result of emulating the biomass production process, wherein the model process is a set of rules for controlling the biomass production process; c) determining operation parameters of the selected control tools using a trained model process based on the results of emulating the biomass production process; d) producing biomass using the selected control tools that work with certain parameters.

15. The method of microbial biomass production according to alternative 14, wherein the microorganism is a methanotrophic microorganism.

16. The method of microbial biomass production according to any one of alternatives 14-15, wherein the parameters of the biomass production process comprise: productivity of biomass production; the proportion of crude protein containing in the biomass; specific power consumption; or the specific consumption of the resources used to produce biomass.

17. The method of microbial biomass production according to any one of alternatives 14-16, wherein the result of the emulating the biomass production process comprises: information about the resources of the management tools used to produce the specified biomass; or parameters of the biomass production process.

18. The method of microbial biomass production according to any one of alternatives 14-17, wherein the control tools comprise: a flow of gas mixture regulating tool, configured to control the gas mixture used in the biomass production process; a biomass controlling tool, configured to control the parameters of the produced biomass; or an aeration tool, configured to ensure the mass transfer of the nutrient medium gas components and culture liquid oxygen.

19. The method of microbial biomass production according to alternative 18, wherein the gas mixture is controlled by at least: changing the composition of the gas mixture by changing the concentration of the components of the gas mixture; changing the temperature of the gas mixture; or changing the pressure of the gas mixture.

20. The method of microbial biomass production according to alternative 19, wherein at least two components of the gas mixture act as: nitrogen; oxygen; natural gas; carbon dioxide; or air.

21. The method of microbial biomass production according to alternative 20, wherein the gas mixture is controlled in a predetermined explosion-proof area in such a way as to ensure the maximum permissible concentration of oxygen.

22. The method of microbial biomass production according to alternative 21, wherein the characteristics of the increased oxygen flow zone according to the Gibbs-Rosebum diagram act as parameters of the gas mixture flow control device operation.

23. The method of microbial biomass production according to alternative 22, wherein the characteristics of the increased oxygen flow zone according to the Gibbs-Rosebum diagram comprise concentrations of nitrogen at 81.9%, methane at 6.0%, and oxygen at 12.1%.

24. The method of microbial biomass production according to any one of alternatives 18-23, wherein the biomass control comprises: changing the biomass temperature; or changing the pH of the biomass.

25. The method of microbial biomass production according to any one of alternatives 18-24, wherein the aeration control is a change in the variable aeration, which is reached at the maximum value of the enzyme that is the activator of molecular oxygen in the ventilation zone, followed by oxidation of the organic substrate.

26. The method of microbial biomass production according to any one of alternatives 14-25, further comprising a training tool configured to retrain the model process in such a way that at least: for the specified parameters of biomass production, the specified biomass will be produced with less usage of management resources; or for the specified resources of the biomass production management tools, more optimal parameters for producing the specified biomass will be selected.

In conclusion, it should be noted that the information provided in the description is an example, and does not limit the scope of the present disclosure defined by the claims.

What is claimed is:

1. A microbial biomass production system, containing:
a) a process emulation tool configured to emulate in a virtual environment of a biomass production process with specified parameters, where the virtual environment is a model of industrial production of microorganisms;
b) a selection tool configured for:
selecting at least one control tool of the biomass production process from a list of control tools using a trained model process based on the result of emulation of the biomass production process, wherein the model process includes a set of rules for controlling of the biomass production process;
determining operation parameters of the selected control tool using a trained model process based on the results of emulation of the biomass production process; and
c) a production tool configured to produce biomass using the selected control tool that operates with certain parameters.

2. The microbial biomass production system according to claim 1, wherein the microorganism is a methanotrophic microorganism.

3. The microbial biomass production system according to claim 1, wherein the parameters of the biomass production process comprise:
productivity of biomass production;
the proportion of crude protein contained in the biomass;
specific power consumption; and
the specific consumption of the resources used to produce biomass.

4. The microbial biomass production system according to claim 1, wherein the result of the emulation of the biomass production process comprises:
information about the resources of the management tools used to produce the specified biomass; or
parameters of the biomass production process.

5. The microbial biomass production system according to claim 1, wherein the control tools comprise:
a flow of gas mixture regulating tool, configured to control the gas mixture used in the biomass production process;
a biomass controlling tool, configured to control the parameters of the resulting biomass; or
an aeration tool, configured to ensure the mass transfer of nutrient medium gas components and culture liquid oxygen.

6. The microbial biomass production system according to claim 5, wherein the gas mixture is controlled by at least:
changing the composition of the gas mixture by changing the concentration of the components of the gas mixture;
changing the temperature of the gas mixture; or
changing the pressure of the gas mixture.

7. The microbial biomass production system according to claim 6, wherein at least two components of the gas mixture act as: nitrogen; oxygen; natural gas; carbon dioxide; or air.

8. The microbial biomass production system according to claim 7, wherein the gas mixture is controlled in a predetermined explosion-proof area in such a way as to ensure the maximum permissible concentration of oxygen.

9. The microbial biomass production system according to claim 8, wherein the characteristics of the increased oxygen flow zone according to the Gibbs-Rosebum diagram act as parameters of the gas mixture flow control device operation.

10. The microbial biomass production system according to claim 9, wherein the characteristics of the increased oxygen flow zone according to the Gibbs-Rosebum diagram comprise nitrogen at 81.9%, methane at 6.0%, and oxygen at 12.1%.

11. The microbial biomass production system according to claim 5, wherein the biomass control comprise:
changing the biomass temperature;
changing the pH of the biomass.

12. The microbial biomass production system according to claim 5, wherein the aeration control is a change in the variable aeration, which is reached at the maximum value of the enzyme that is the activator of molecular oxygen in the ventilation zone, followed by oxidation of the organic substrate.

13. The microbial biomass production system according to claim 1, further comprising a training tool configured to retrain the model process in such a way that at least:
for the specified parameters of biomass production, the specified biomass will be produced with less usage of management resources; or
for the specified resources of the biomass production management tools, more optimal parameters for producing the specified biomass will be selected.

14. A method of microbial biomass production, comprising:
a) emulating in a virtual environment a biomass production process with specified parameters, wherein the virtual environment is a model of industrial production of microorganisms;
b) selecting at least one control tool of the biomass production process from the list of control tools using a trained model process based on the result of emulating the biomass production process, wherein the model process is a set of rules for controlling the biomass production process;
c) determining operation parameters of the selected control tools using a trained model process based on the results of emulating the biomass production process;
d) producing biomass using the selected control tools that work with certain parameters.

15. The method of microbial biomass production according to claim 14, wherein the microorganism is a methanotrophic microorganism.

16. The method of microbial biomass production according to claim 14, wherein the parameters of the biomass production process comprise:
productivity of biomass production;
the proportion of crude protein containing in the biomass;
specific power consumption; or
the specific consumption of the resources used to produce biomass.

17. The method of microbial biomass production according to claim 14, wherein the result of the emulating the biomass production process comprises:
information about the resources of the management tools used to produce the specified biomass; or
parameters of the biomass production process.

18. The method of microbial biomass production according to claim 14, wherein the control tools comprise:
a flow of gas mixture regulating tool, configured to control the gas mixture used in the biomass production process;
a biomass controlling tool, configured to control the parameters of the produced biomass; or
an aeration tool, configured to ensure the mass transfer of the nutrient medium gas components and culture liquid oxygen.

19. The method of microbial biomass production according to claim 18, wherein the gas mixture is controlled by at least:
changing the composition of the gas mixture by changing the concentration of the components of the gas mixture;
changing the temperature of the gas mixture; or
changing the pressure of the gas mixture.

20. The method of microbial biomass production according to claim 19, wherein at least two components of the gas mixture act as: nitrogen; oxygen; natural gas; carbon dioxide; or air.

21. The method of microbial biomass production according to claim 20, wherein the gas mixture is controlled in a predetermined explosion-proof area in such a way as to ensure the maximum permissible concentration of oxygen.

22. The method of microbial biomass production according to claim 21, wherein the characteristics of the increased oxygen flow zone according to the Gibbs-Rosebum diagram act as parameters of the gas mixture flow control device operation.

23. The method of microbial biomass production according to claim 22, wherein the characteristics of the increased oxygen flow zone according to the Gibbs-Rosebum diagram comprise concentrations of nitrogen at 81.9%, methane at 6.0%, and oxygen at 12.1%.

24. The method of microbial biomass production according to claim 18, wherein the biomass control comprises:
changing the biomass temperature; or
changing the pH of the biomass.

25. The method of microbial biomass production according to claim 18, wherein the aeration control is a change in the variable aeration, which is reached at the maximum value of the enzyme that is the activator of molecular oxygen in the ventilation zone, followed by oxidation of the organic substrate.

26. The method of microbial biomass production according to claim 14, further comprising a training tool configured to retrain the model process in such a way that at least:
for the specified parameters of biomass production, the specified biomass will be produced with less usage of management resources; or
for the specified resources of the biomass production management tools, more optimal parameters for producing the specified biomass will be selected.
